(19) United States
(12) Patent Application Publication
(10) Pub. No.: US 2023/0073802 A1
(43) Pub. Date: Mar. 9, 2023

(54) COMPOSITIONS AND METHODS RELATED TO CANNABINOID ANIONS

(71) Applicant: NATURAL EXTRACTION SYSTEMS, LLC, Boulder, CO (US)

(72) Inventor: Douglas G. Metcalf, Boulder, CO (US)

(73) Assignee: NATURAL EXTRACTION SYSTEMS, LLC, Boulder, CO (US)

(21) Appl. No.: 17/797,090

(22) PCT Filed: Feb. 3, 2021
(86) PCT No.: PCT/US2021/016488

§ 371 Date: Aug. 2, 2022

Related U.S. Application Data
(60) Provisional application No. 62/971,808, filed on Feb. 7, 2020.
(60) Provisional application No. 63/059,829, filed on Jul. 31, 2020.

(51) Int. Cl. A61K 31/352; A61K 31/05; A61K 9/08; A61K 47/10

(52) U.S. Cl. CPC A61K 31/352; A61K 31/05; A61K 9/08; A61K 47/10

(57) ABSTRACT

Various aspects of the disclosure relate to compositions comprising a liquid phase that comprises a cannabinoid anion, wherein the cannabinoid anion is the conjugate base of a cannabinoid molecule, and the cannabinoid molecule has an acid dissociation constant in water of at least 50 femtomolar and no greater than 50 nanomolar for conversion of the cannabinoid molecule into the cannabinoid anion.

### CROSS-REFERENCE TO RELATED APPLICATIONS

This patent document claims priority to U.S. Provisional Patent Application No. 62/971,808, filed Feb. 7, 2020, and U.S. Provisional Patent Application No. 63/059,829, filed Jul. 31, 2020, both of which are incorporated by reference in their entirety.

### BACKGROUND

Methods to improve the solubility, bioavailability, and pharmacokinetics of cannabinoids are desirable.

### SUMMARY

Cannabinoids are generally insoluble in water and glycerol. Cannabinoids can nevertheless be dissolved in water and glycerol by conversion from their conventional molecular form into anions under alkaline conditions as described, for example, in U.S. Pat. Nos. 10,555,914 and 10,609,944. The resultant compositions display improved bioavailability and pharmacokinetics, for example, because the negative charges of cannabinoid anions repel each other to maximize surface area, and because the reconversion of cannabinoid anions into cannabinoid molecules allows rapid partitioning from a hydrophilic vehicle into hydrophobic tissues. This disclosure describes specific formulations related to cannabinoid anions and their methods of use.

### DETAILED DESCRIPTION

Various aspects of the disclosure relate to a composition, comprising a liquid phase that comprises ethanol at a concentration of at least 4 molar, ethoxide at a concentration of at least 40 nanomolar, a cannabinoid anion at a concentration of at least 1 millimolar, a cannabinoid molecule at a concentration of at least 500 picomolar, and a cation at a concentration of at least 500 micromolar, wherein: the liquid phase is miscible with water; the ethoxide, the cannabinoid anion, the cannabinoid molecule, and the cation are solutes that are dissolved in the liquid phase; the cannabinoid molecule has an acid dissociation constant in water of at least 50 femtomolar and no greater than 50 nanomolar for conversion of the cannabinoid molecule into the cannabinoid anion; the liquid phase comprises the cannabinoid anion at a greater molar concentration than the cannabinoid molecule; the cation is a metal cation or an ammonium cation; and the liquid phase comprises the cation and the cannabinoid anion at a molar ratio of at least 1:3 (cation:cannabinoid anion).

“Comprising” and “comprise(s)” refer to open sets such that a composition that comprises ethanol can also comprise water.

“Cannabinoid molecule” refers to a cannabinoid chemical species that lacks a net charge, and “cannabinoid anion” refers to a cannabinoid chemical species that comprises a net negative charge. Various cannabinoid molecules and cannabinoid anions are described in PCT Patent Application Publication No. WO2020123809, PCT Patent Application Publication No. WO2020123976, PCT Patent Application Publication No. WO2020264199, U.S. Pat. Nos. 10,555,914, and 10,609,944, which are incorporated by reference for the chemical species that they disclose.

“Dissolved” refers to a solute that is solvated in a liquid phase; a chemical species that is present within a phase that is dispersed within a liquid phase, such as the dispersed phase of an emulsion, is not dissolved in the liquid phase; a chemical species that is non-covalently bound to any chemical species that is a solid in the absence of a solvent, such as a cyclodextrin, is not dissolved in a liquid phase.

“Ammonium cation” refers to both ammonium and aminium cations.

In some specific embodiments, the liquid phase comprises the ethanol at a concentration of at least 10 molar and the ethoxide at a concentration of at least 100 nanomolar. In some very specific embodiments, the liquid phase comprises the ethanol at a concentration of at least 15 molar and the ethoxide at a concentration of at least 150 nanomolar.

In some embodiments, the liquid phase comprises water at a concentration of at least 1 molar; the liquid phase comprises the water and the ethanol at a mass ratio of at least 1:100 and no greater than 2:1 (water:ethanol); and the liquid phase comprises hydroxide at a concentration of at least 10 nanomolar and no greater than one-tenth of the concentration of the water.

In some embodiments, the liquid phase comprises propane-1,2-diol at a concentration of at least 1 molar; and the liquid phase comprises 1-hydroxypropane-2-oxide and 2-hydroxypropane-1-oxide at a combined concentration of at least 10 nanomolar and no greater than one-tenth of the concentration of the propane-1,2-diol.

In some embodiments, the liquid phase comprises propane-1,2,3-triol (glycerol) at a concentration of at least 1 molar; and the liquid phase comprises 1,3-dihydroxypropane-2-oxide and 2,3-dihydroxypropane-1-oxide at a combined concentration of at least 10 nanomolar and no greater than one-tenth of the concentration of the propane-1,2,3-triol.

In some embodiments, the composition comprises a solid phase that comprises one or more of butane-1,2,3,4-tetrol, pentane-1,2,3,4,5-pentol, hexane-1,2,3,4,5,6-hexol, cyclohexane-1,2,3,4,5,6-hexol, and heptane-1,2,3,4,5,6,7-heptol, wherein the composition comprises the solid phase and the liquid phase at a mass ratio of at least 3:1 and no greater than 100:1 (solid phase:liquid phase). “Butane-1,2,3,4-tetrol” comprises, for example, erythritol and threitol. “Pentane-1,2,3,4,5-pentol” comprises, for example arabitol, ribitol, and xylitol. “Hexane-1,2,3,4,5,6-hexol” comprises, for example, sorbitol, fucitol, mannitol, galactitol, and iditol. “Cyclohexane-1,2,3,4,5,6-hexol” comprises, for example, inositol. “Heptane-1,2,3,4,5,6,7-heptol” comprises, for example, volemitol.

Various aspects of the disclosure relate to a composition, comprising a liquid phase that comprises water at a concentration of at least 35 molar, hydroxide at a concentration of at least 100 nanomolar and no greater than 100 millimolar, a cannabinoid anion at a concentration of at least 10 micromolar and no greater than 10 millimolar, a cannabinoid molecule at a concentration of at least 100 picomolar and no greater than 10 micromolar, and a cation at a concentration of at least 5 micromolar, wherein: the hydroxide, the cannabinoid anion, the cannabinoid molecule, and the cation are solutes that are dissolved in the liquid phase; the cannabinoid molecule has an acid dissociation constant in water of at least 50 femtomolar and no greater than 50 nanomolar for conversion of the cannabinoid molecule into the cannabinoid anion; the cation is a metal cation or an ammonium cation; and the liquid phase comprises the cation and the cannabinoid anion at a molar ratio of at least 1:3 and no greater than 10,000:1 (cation:cannabinoid anion). In some specific embodiments, the liquid phase comprises the water at a concentration of at least 50 molar.

In some embodiments, the liquid phase comprises ethanol at a concentration of at least 500 micromolar and ethoxide at a concentration of at least 5 picomolar.

In some embodiments, the liquid phase comprises a sugar alcohol at a concentration of at least 1 millimolar, and either: the sugar alcohol is propane-1,2-diol, and the liquid phase comprises 1-hydroxypropane-2-oxide and 2-hydroxypropane-1-oxide at a combined concentration of at least 10 picomolar and no greater than one-tenth of the concentration of the sugar alcohol; the sugar alcohol is propane-1,2,3-triol, and the liquid phase comprises 1,3-dihydroxypropane-2-oxide and 2,3-dihydroxypropane-1-oxide at a combined concentration of at least 10 picomolar and no greater than one-tenth of the concentration of the sugar alcohol; the sugar alcohol is butane-1,2,3,4-tetrol, and the liquid phase comprises 1,3,4-trihydroxybutane-2-oxide and 2,3,4-trihydroxybutane-1-oxide at a combined concentration of at least 10 picomolar and no greater than one-tenth of the concentration of the sugar alcohol; the sugar alcohol is pentane-1,2,3,4,5-pentol, and the liquid phase comprises 1,2,4,5-tetrahydroxypentane-3-oxide, 1,3,4,5-tetrahydroxypentane-2-oxide, and 2,3,4,5-tetrahydroxypentane-1-oxide at a combined concentration of at least 10 picomolar and no greater than one-tenth of the concentration of the sugar alcohol; the sugar alcohol is hexane-1,2,3,4,5,6-hexol, and the liquid phase comprises 1,2,4,5,6-pentahydroxyhexane-3-oxide, 1,3,4,5,6-pentahydroxyhexane-2-oxide, and 2,3,4,5,6-pentahydroxyhexane-1-oxide at a combined concentration of at least 10 picomolar and no greater than one-tenth of the concentration of the sugar alcohol; the sugar alcohol is cyclohexane-1,2,3,4,5,6-hexol, and the liquid phase comprises 2,3,4,5,6-pentahydroxycyclohexane-1-oxide at a concentration of at least 10 picomolar and no greater than one-tenth of the concentration of the sugar alcohol; or the sugar alcohol is heptane-1,2,3,4,5,6,7-heptol, and the liquid phase comprises 1,2,3,5,6,7-hexahydroxyheptane-4-oxide, 1,2,4,5,6,7-hexahydroxyheptane-3-oxide, 1,3,4,5,6,7-hexahydroxyheptane-2-oxide, and 2,3,4,5,6,7-hexahydroxyheptane-1-oxide at a combined concentration of at least 10 picomolar and no greater than one-tenth of the concentration of the sugar alcohol.

Various aspects of the disclosure relate to a composition, comprising a liquid phase that comprises propane-1,2-diol at a concentration of at least 1 molar, 1-hydroxypropane-2-oxide and 2-hydroxypropane-1-oxide at a combined concentration of at least 10 nanomolar and no greater than 100 millimolar, a cannabinoid anion at a concentration of at least 1 millimolar and no greater than 500 millimolar, a cannabinoid molecule at a concentration of at least 100 nanomolar and no greater than 50 millimolar, and a cation at a concentration of at least 500 micromolar, wherein: the liquid phase is miscible with water; the 1-hydroxypropane-2-oxide, the 2-hydroxypropane-1-oxide, the cannabinoid anion, the cannabinoid molecule, and the cation are solutes that are dissolved in the liquid phase; the cannabinoid molecule has an acid dissociation constant in water of at least 50 femtomolar and no greater than 50 nanomolar for conversion of the cannabinoid molecule into the cannabinoid anion; the liquid phase comprises the cannabinoid anion at a greater molar concentration than the cannabinoid molecule; the cation is a metal cation or an ammonium cation; and the liquid phase comprises the cation and the cannabinoid anion at a molar ratio of at least 1:3 and no greater than 1000:1 (cation:cannabinoid anion). In some specific embodiments, the liquid phase comprises the propane-1,2-diol at a concentration of at least 10 molar, and the liquid phase comprises the 1-hydroxypropane-2-oxide and the 2-hydroxypropane-1-oxide at a combined concentration of at least 100 nanomolar.

Various aspects of the disclosure relate to a composition, comprising a liquid phase that comprises propane-1,2,3-triol at a concentration of at least 1 molar, 1,3-dihydroxypropane-2-oxide and 2,3-dihydroxypropane-1-oxide at a combined concentration of at least 10 nanomolar and no greater than 100 millimolar, a cannabinoid anion at a concentration of at least 1 millimolar and no greater than 500 millimolar, a cannabinoid molecule at a concentration of at least 100 nanomolar and no greater than 50 millimolar, and a cation at a concentration of at least 500 micromolar, wherein: the liquid phase is miscible with water; the 1,3-dihydroxypropane-2-oxide, the 2,3-dihydroxypropane-1-oxide, the cannabinoid anion, the cannabinoid molecule, and the cation are solutes that are dissolved in the liquid phase; the cannabinoid molecule has an acid dissociation constant in water of at least 50 femtomolar and no greater than 50 nanomolar for conversion of the cannabinoid molecule into the cannabinoid anion; the liquid phase comprises the cannabinoid anion at a greater molar concentration than the cannabinoid molecule; the cation is a metal cation or an ammonium cation; and the liquid phase comprises the cation and the cannabinoid anion at a molar ratio of at least 1:3 and no greater than 1000:1 (cation:cannabinoid anion). In some specific embodiments, the liquid phase comprises the propane-1,2,3-triol at a concentration of at least 10 molar, and the liquid phase comprises the 1,3-dihydroxypropane-2-oxide and the 2,3-dihydroxypropane-1-oxide at a combined concentration of at least 100 nanomolar.

In some embodiments, the liquid phase comprises ethanol, water, ethoxide, and hydroxide; and the liquid phase comprises the ethanol at a greater concentration by mass than the water.

In some embodiments, the liquid phase comprises ethanol at a concentration of at least 3 molar, and the liquid phase comprises ethoxide at a concentration of at least 30 nanomolar and no greater than one-tenth of the concentration of the ethanol.

In some embodiments, the liquid phase comprises water at a concentration of at least 10 millimolar and no greater than 5 molar.

In some embodiments, the cannabinoid anion has the general structure I, II, or III.

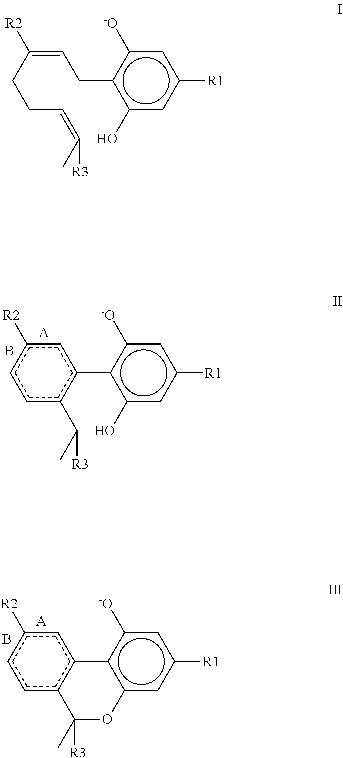

The dotted lines in general structures II and III depict the bonding pattern of either cyclohexane, phenyl, or a cyclohexene that comprises exactly one double bond, which occurs at either A or B.

In some embodiments, R1 is selected from hydro; a straight or branched C1-C12 alkyl that is optionally substituted with hydroxy, phenyl, a cycloalkyl, or a halogen; a straight or branched C2-C12 alkenyl that is optionally substituted with hydroxy, phenyl, a cycloalkyl, or a halogen; and a straight or branched C2-C12 alkynyl that is optionally substituted with hydroxy, phenyl, a cycloalkyl, or a halogen.

“Straight or branched C1-C12 alkyl” refers to an unbranched or branched hydrocarbon chain having 1, 2, 3, 4, 5, 6, 7, 8, 9, 10, 11, or 12 carbon atoms, wherein all carbon-carbon bonds in the hydrocarbon chain are single bonds.

“Substituted with hydroxy, phenyl, a cycloalkyl, or a halogen” refers to the substitution of at least one hydrogen atom of a hydrocarbon chain with hydroxy, phenyl, a cycloalkyl, or a halogen.

“Halogen” refers to F, Cl, Br, and I.

“Cycloalkyl” refers to cyclopropyl, cyclobutyl, cyclopentyl, cyclohexyl, cycloheptyl, and adamantyl. When a hydrocarbon chain is substituted with a cycloalkyl, then either (i) a single hydrogen atom of the hydrocarbon chain is substituted with the cycloalkyl such that the cycloalkyl does not include any carbon atom of the hydrocarbon chain, or (ii) two hydrogen atoms of the hydrocarbon chain are substituted with the cycloalkyl such that the cycloalkyl comprises one or more carbon atoms of the hydrocarbon chain.

“Straight or branched C2-C12 alkenyl” refers to an unbranched or branched hydrocarbon chain having 2, 3, 4, 5, 6, 7, 8, 9, 10, 11, or 12 carbon atoms, wherein at least one carbon-carbon bond in the hydrocarbon chain is a double bond and no carbon-carbon bond in the hydrocarbon chain is a triple bond.

“Straight or branched C2-C12 alkynyl” refers to an unbranched or branched hydrocarbon chain having 2, 3, 4, 5, 6, 7, 8, 9, 10, 11, or 12 carbon atoms, wherein at least one carbon-carbon bond in the hydrocarbon chain is a triple bond.

In some specific embodiments, R1 is hydro; methyl; ethyl; propyl; butyl; pentyl; hexyl; heptyl; octyl; nonyl; decyl; prop-2-yl; but-2-yl; pent-2-yl; hex-2-yl; hept-2-yl; octan-2-yl; nonan-2-yl; decan-2-yl; 2-methylpropyl; 2-methylbutyl; 2-methylpentyl; 2-methylhexyl; 2-methylheptyl; 2-methyloctyl; 2-methylnonyl; 2-methyldecyl; 2-methylprop-2-yl; 2-methylbut-2-yl; 2-methylpent-2-yl; 2-methylhex-2-yl; 2-methylhept-2-yl; 2-methyloctan-2-yl; 2-methylnonan-2-yl; 2-methyldecan-2-yl; 3-methylbut-2-yl; 3-methylpent-2-yl; 3-methylhex-2-yl; 3-methylhept-2-yl; 3-methyloctan-2-yl; 3-methylnonan-2-yl; 3-methyldecan-2-yl; 2,3-dimethylbut-2-yl; 2,3-dimethylpent-2-yl; 2,3-dimethylhex-2-yl; 2,3-dimethylhept-2-yl; 2,3-dimethyloctan-2-yl; 2,3-dimethylnonan-2-yl; 2,3-dimethyldecan-2-yl; cyclopropyl; 1-methylcyclopropyl; 1-ethylcyclopropyl; 1-propylcyclopropyl; 1-butylcyclopropyl; 1-pentylcyclopropyl; 1-hexylcyclopropyl; 1-heptylcyclopropyl; 1-octylcyclopropyl; 1-nonylcyclopropyl; cyclobutyl; 1-methylcyclobutyl; 1-ethylcyclobutyl; 1-propylcyclobutyl; 1-butylcyclobutyl; 1-pentylcyclobutyl; 1-hexylcyclobutyl; 1-heptylcyclobutyl; 1-octylcyclobutyl; cyclopentyl; 1-methylcyclopentyl; 1-ethylcyclopentyl; 1-propylcyclopentyl; 1-butylcyclopentyl; 1-pentylcyclopentyl; 1-hexylcyclopentyl; 1-heptylcyclopentyl; cyclohexyl; 1-methylcyclohexyl; 1-ethylcyclohexyl; 1-propylcyclohexyl; 1-butylcyclohexyl; 1-pentylcyclohexyl; 1-hexylcyclohexyl; ethenyl; prop-1-enyl; but-1-enyl; pent-1-enyl; hex-1-enyl; hept-1-enyl; octan-1-enyl; nonan-1-enyl; decan-1-enyl; ethynyl; prop-1-ynyl; but-1-ynyl; pent-1-ynyl; hex-1-ynyl; hept-1-ynyl; octan-1-ynyl; nonan-1-ynyl; decan-1-ynyl; 2-phenylethyl; 2-phenylprop-2-yl; adamant-1-yl; adamant-2-yl; 6-halohex-2-enyl; 6-halohex-2-ynyl; or 2-methyl-6-halohex-2-yl. “Halo” refers to fluoro, chloro, bromo, or iodo. In some very specific embodiments, R1 is propyl or pentyl.

In some embodiments, R2 is selected from hydro; methyl; methylidene, hydroxy; hydroxymethyl; fluoromethyl; chloromethyl; bromomethyl; iodomethyl; oxo; formyl; methoxycarbonyl; ethoxycarbonyl; and (2-propoxy)carbonyl. In some specific embodiments, either (i) R2 is hydro, methyl, hydroxy, or hydroxymethyl; or (ii) each of the cannabinoid anion has the general structure II or III, R2 is oxo, and the dotted lines in general structures II and III depict the bonding pattern of cyclohexane. In some very specific embodiments, R2 is methyl.

In some embodiments, R3 is selected from a hydro; straight C1-C3 alkyl that is optionally substituted with hydroxy or a halogen; a straight C1-C3 alkenyl that is optionally substituted with hydroxy or a halogen; and a C2-C3 alkynyl that is optionally substituted with hydroxy or a halogen.

“Straight C1-C3 alkyl” refers to an unbranched hydrocarbon chain having 1, 2, or 3 carbon atoms, wherein all carbon-carbon bonds in the hydrocarbon chain are single bonds.

“Substituted with hydroxy or a halogen” refers to the substitution of at least one hydrogen atom of a hydrocarbon chain with hydroxy or a halogen.

“Straight C1-C3 alkenyl” encompasses both methylidene and an unbranched hydrocarbon chain having 2 or 3 carbon atoms, wherein at least one carbon-carbon bond in the unbranched hydrocarbon chain having 2 or 3 carbon atoms is a double bond.

“C2-C3 alkynyl” refers to an unbranched hydrocarbon chain having 2 or 3 carbon atoms, wherein exactly one carbon-carbon bond in the hydrocarbon chain is a triple bond.

In some specific embodiments, either: (i) the cannabinoid anion has the general structure I or III, and R3 is methyl; or (ii) the cannabinoid anion has the general structure II, and R3 is methylidene.

In some specific embodiments, the cannabinoid anion has the general structure II or III; and R3 is methyl, 3-hydroxypropyl, 3-hydroxyprop-1-enyl, or 3-hydroxyprop-1-ynyl.

In some very specific embodiments, the cannabinoid anion is 2-geranyl-3-hydroxy pentylphenolate.

In some very specific embodiments, the cannabinoid anion is 2-geranyl-3-hydroxy propylphenolate.

In some very specific embodiments, the cannabinoid anion is 3-hydroxy-2-(6-isopropenyl methylcyclohex-2-enyl)-5-pentylphenolate.

In some very specific embodiments, the cannabinoid anion is 3-hydroxy-2-(6-isopropenyl-3-methylcyclohex-2-enyl)-5-propylphenolate.

In some very specific embodiments, the cannabinoid anion is 6,6,9-trimethyl-3-pentyl-6a,7,8,10a-tetrahydro-6H-benzo[c]chromene-1-oxide.

In some very specific embodiments, the cannabinoid anion is 6,6,9-trimethyl-3-propyl-6a,7,8,10a-tetrahydro-6H-benzo[c]chromene-1-oxide.

In some very specific embodiments, the cannabinoid anion is 6,6,9-trimethyl-3-pentyl-6H-benzo[c]chromene-1-oxide.

In some very specific embodiments, the cannabinoid anion is 6,6,9-trimethyl-3-propyl-6H-benzo[c]chromene-1-oxide.

In some specific embodiments, the cation is lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); copper (II) cation (“Cu++”); ammonium (“NH4+”); protonated ethanolamine; choline; protonated lysine; protonated arginine; or protonated sphingosine. In some very specific embodiments, the cation is sodium cation. In some very specific embodiments, the cation is potassium cation.

In some preferred embodiments, the cannabinoid molecule has a solubility in the liquid phase that is less than the concentration of the cannabinoid anion in the liquid phase.

Various aspects of the disclosure relate to a composition prepared from a starting composition according to any of the preceding embodiments, comprising a liquid phase that comprises a cannabinoid molecule and a cation, wherein: the cannabinoid molecule and the cation are solutes that are dissolved in the liquid phase; the composition is prepared by contacting the starting composition with a Brønsted acid to convert the cannabinoid anion of the starting composition into the cannabinoid molecule; the liquid phase comprises the cation and the cannabinoid molecule at a molar ratio of at least 1:3 (cation:cannabinoid molecule); and the cannabinoid molecule has a solubility in the liquid phase that is less than the concentration of the cannabinoid molecule that is dissolved in the liquid phase such that the liquid phase is supersaturated with the cannabinoid molecule.

Various aspects of the disclosure relate to a composition described anywhere in this patent document for use as a medicament.

In some embodiments, the composition is formulated to convert the cannabinoid anion into the cannabinoid molecule ex vivo prior to administering the composition to a subject.

In some embodiments, the composition is formulated to convert the cannabinoid anion into the cannabinoid molecule in situ subsequent to administering the composition to a subject.

In some embodiments, the composition is formulated for oral administration to a subject; the composition is formulated to allow the conversion of the cannabinoid anion into the cannabinoid molecule before the cannabinoid anion reaches the stomach of the subject; and the composition is formulated to allow absorption of the cannabinoid molecule by the epithelial lining of the gastrointestinal tract between the lips and the stomach, excluding the stomach and the outer surfaces of the lips, and including the esophagus and the inner surfaces of the lips.

In some embodiments, the composition is formulated for topical administration to a subject.

Various aspects of the disclosure relate to a method to administer a cannabinoid, comprising providing a composition according to any of the preceding embodiments, and administering the composition to a subject.

In some embodiments, the method comprises converting the cannabinoid anion into the cannabinoid molecule ex vivo prior to the administering.

In some embodiments, the method comprises converting the cannabinoid anion into the cannabinoid molecule in situ subsequent to the administering.

In some embodiments, the administering is oral administering; the composition is formulated to allow the conversion of the cannabinoid anion into the cannabinoid molecule before the cannabinoid anion reaches the stomach of the subject; and the composition is formulated to allow absorption of the cannabinoid molecule by the epithelial lining of the gastrointestinal tract between the lips and the stomach, excluding the stomach and the outer surfaces of the lips, and including the esophagus and the inner surfaces of the lips.

In some embodiments, the administering is topical administering.

In some specific embodiments, the subject is a rodent, lagomorph, feline, canine, porcine, ovine, caprine, lama, vicugna, bovine, equine, or primate. In some very specific embodiments, the composition is formulated for veterinary use. In some very specific embodiments, the subject is human.

In some embodiments, the composition is effective to prophylactically prevent or treat muscle cramping, muscle spasms, restless-legs syndrome, nystagmus, a dyskinetic movement disorder, tremor, seizures, epilepsy, muscular dystrophy, or inclusion body myositis. In some embodiments, the composition is effective to either arrest or reduce the severity of an active seizure. In some embodiments, the composition is effective to reduce blood pressure. In some embodiments, the composition is effective to prophylactically prevent or treat prehypertension or hypertension. In some embodiments, the composition is effective to reduce intraocular pressure. In some embodiments, the composition is effective to treat attention deficit hyperactivity disorder (“ADHD”), autism or an autism spectrum disorder, Asperger syndrome, fragile X syndrome, Down syndrome, a pervasive developmental disorder not otherwise specified (“PDD-NOS”), a childhood disintegrative disorder, or Tourette's syndrome. In some embodiments, the composition is effective to treat anxiety, post-traumatic stress disorder (“PTSD”), depression, bipolar disorder, obsessive-compulsive disorder, schizophreniform disorder, schizophrenia, or psychosis. In some embodiments, the composition is effective to treat pain or inflammation. In some embodiments, the composition is effective to treat asthma. In some embodiments, the composition is effective to treat an autoimmune disorder. In some embodiments, the composition is effective to treat arthritis, ankylosing spondylitis, an inflammatory autoimmune-mediated arthritis, rheumatoid arthritis, psoriatic arthritis, psoriasis, plaque psoriasis, lupus, Sjogren's syndrome, inflammatory bowel disease, Crohn's disease, or ulcerative colitis. In some embodiments, the composition is effective to treat a neurodegenerative disease or neuropathy. In some embodiments, the composition is effective to treat Parkinson's Disease, and treating the Parkinson's Disease comprises treating Parkinsonian tremor. In some embodiments, the composition is effective to treat multiple sclerosis, mild cognitive impairment, Alzheimer's Disease, amyotrophic lateral sclerosis (“ALS”), or Huntington's disease. In some embodiments, the composition is effective to treat obesity, metabolic syndrome, or diabetes mellitus. In some embodiments, the composition is effective to treat a viral infection or a bacterial infection. In some embodiments, the composition is effective to treat an infection caused by Escherichia coli, Klebsiella pneumoniae, Pseudomonas aeruginosa, Acinetobacter baumannii, Moraxella catarrhalis, Legionella pneumophila, Staphylococcus aureus, Streptococcus pneumoniae, Enterococcus faecium, Clostridioides difficile, Mycobacterium tuberculosis, Neisseria gonorrhoeae, Cutibacterium acnes, or COVID-19.

“Treat” refers to at least one of: to cure a health condition; to increase the probability that a health condition will be cured; to shorten the time over which a health condition is cured; to increase the probability that the time necessary to cure a health condition will be shortened; to decrease the severity of a health condition; to increase the probability that the severity of a health condition will decrease; to shorten the time over which the severity of a health condition is decreased; to increase the probability that the time necessary to decrease the severity of a health condition will be shortened; to inhibit a health condition from worsening; to increase the probability that a health condition will not worsen; to delay the worsening of a health condition; to increase the probability that the worsening of a health condition will be delayed; to inhibit the occurrence or recurrence of a health condition; to decrease the probability that a health condition will occur or reoccur; to delay the onset of a health condition; to increase the probability that the onset of a health condition will be delayed; to alleviate at least one symptom of a health condition; to increase the probability that at least one symptom of a health condition will be alleviated; to shorten the time over which at least one symptom of a health condition is alleviated; to increase the probability that the time necessary to alleviate at least one symptom of a health condition will be shortened; to decrease the severity of at least one symptom of a health condition; to increase the probability that the severity of at least one symptom of a health condition will be decreased; to shorten the time over which the severity of at least one symptom of a health condition is decreased; to increase the probability that the time necessary to decrease the severity of at least one symptom of a health condition will be shortened; to inhibit at least one symptom of a health condition from worsening; to increase the probability that at least one symptom of a health condition will not worsen; to delay the worsening of at least one symptom of a health condition; to increase the probability that the worsening of at least one symptom of a health condition will be delayed; to inhibit at least one symptom of a health condition from occurring or reoccurring; to decrease the probability that at least one symptom of a health condition will occur or reoccur; to delay the onset of at least one symptom of a health condition; and to increase the probability that the onset of at least one symptom of a health condition will be delayed.

What is claimed is:

1. A composition, comprising a liquid phase that comprises ethanol at a concentration of at least 4 molar, ethoxide at a concentration of at least 40 nanomolar, a cannabinoid anion at a concentration of at least 1 millimolar, a cannabinoid molecule at a concentration of at least 500 picomolar, and a cation at a concentration of at least 500 micromolar, wherein:
the liquid phase is miscible with water;
the ethoxide, the cannabinoid anion, the cannabinoid molecule, and the cation are solutes that are dissolved in the liquid phase;
the cannabinoid molecule has an acid dissociation constant in water of at least 50 femtomolar and no greater than 50 nanomolar for conversion of the cannabinoid molecule into the cannabinoid anion;
the liquid phase comprises the cannabinoid anion at a greater molar concentration than the cannabinoid molecule;
the cation is a metal cation or an ammonium cation; and
the liquid phase comprises the cation and the cannabinoid anion at a molar ratio of at least 1:3.

2. The composition of claim 1, wherein the liquid phase comprises the ethanol at a concentration of at least 10 molar and the ethoxide at a concentration of at least 100 nanomolar.

3. The composition of claim 1 or 2, wherein:
the liquid phase comprises water at a concentration of at least 1 molar;
the liquid phase comprises the water and the ethanol at a mass ratio of at least 1:100 and no greater than 2:1; and
the liquid phase comprises hydroxide at a concentration of at least 10 nanomolar and no greater than one-tenth of the concentration of the water.

4. The composition of any one of claims 1-3, wherein the liquid phase comprises propane-1,2-diol at a concentration of at least 1 molar; and the liquid phase comprises 1-hydroxypropane-2-oxide and 2-hydroxypropane-1-oxide at a combined concentration of at least 10 nanomolar and no greater than one-tenth of the concentration of the propane-1,2-diol.

5. The composition of any one of claims 1-3, wherein the liquid phase comprises propane-1,2,3-triol at a concentration of at least 1 molar; and the liquid phase comprises 1,3-dihydroxypropane oxide and 2,3-dihydroxypropane-1-oxide at a combined concentration of at least 10 nanomolar and no greater than one-tenth of the concentration of the propane-1,2,3-triol.

6. The composition of any one of claims 1-3, comprising a solid phase that comprises one or more of butane-1,2,3,4-tetrol, pentane-1,2,3,4,5-pentol, hexane-1,2,3,4,5,6-hexol, cyclohexane-1,2,3,4,5,6-hexol, and heptane-1,2,3,4,5,6,7-heptol, wherein the composition comprises the solid phase and the liquid phase at a mass ratio of at least 3:1 and no greater than 100:1.

7. A composition, comprising a liquid phase that comprises water at a concentration of at least 35 molar, hydroxide at a concentration of at least 100 nanomolar and no greater than 100 millimolar, a cannabinoid anion at a concentration of at least 10 micromolar and no greater than 10 millimolar, a cannabinoid molecule at a concentration of at least 100 picomolar and no greater than 10 micromolar, and a cation at a concentration of at least 5 micromolar, wherein:
the hydroxide, the cannabinoid anion, the cannabinoid molecule, and the cation are solutes that are dissolved in the liquid phase;
the cannabinoid molecule has an acid dissociation constant in water of at least 50 femtomolar and no greater than 50 nanomolar for conversion of the cannabinoid molecule into the cannabinoid anion;
the cation is a metal cation or an ammonium cation; and
the liquid phase comprises the cation and the cannabinoid anion at a molar ratio of at least 1:3 and no greater than 10,000:1.

8. The composition of claim 7, wherein the liquid phase comprises ethanol at a concentration of at least 500 micromolar and ethoxide at a concentration of at least 5 picomolar.

9. The composition of claim 7 or 8, wherein the liquid phase comprises a sugar alcohol at a concentration of at least 1 millimolar, and either:
the sugar alcohol is propane-1,2-diol, and the liquid phase comprises 1-hydroxypropane-2-oxide and 2-hydroxypropane-1-oxide at a combined concentration of at least 10 picomolar and no greater than one-tenth of the concentration of the sugar alcohol;
the sugar alcohol is propane-1,2,3-triol, and the liquid phase comprises 1,3-dihydroxypropane-2-oxide and 2,3-dihydroxypropane-1-oxide at a combined concentration of at least 10 picomolar and no greater than one-tenth of the concentration of the sugar alcohol;
the sugar alcohol is butane-1,2,3,4-tetrol, and the liquid phase comprises 1,3,4-trihydroxybutane-2-oxide and 2,3,4-trihydroxybutane-1-oxide at a combined concentration of at least 10 picomolar and no greater than one-tenth of the concentration of the sugar alcohol;
the sugar alcohol is pentane-1,2,3,4,5-pentol, and the liquid phase comprises 1,2,4,5-tetrahydroxypentane-3-oxide, 1,3,4,5-tetrahydroxypentane-2-oxide, and 2,3,4,5-tetrahydroxypentane-1-oxide at a combined concentration of at least 10 picomolar and no greater than one-tenth of the concentration of the sugar alcohol;
the sugar alcohol is hexane-1,2,3,4,5,6-hexol, and the liquid phase comprises 1,2,4,5,6-pentahydroxyhexane-3-oxide, 1,3,4,5,6-pentahydroxyhexane-2-oxide, and 2,3,4,5,6-pentahydroxyhexane-1-oxide at a combined concentration of at least 10 picomolar and no greater than one-tenth of the concentration of the sugar alcohol;
the sugar alcohol is cyclohexane-1,2,3,4,5,6-hexol, and the liquid phase comprises 2,3,4,5,6-pentahydroxycyclohexane-1-oxide at a concentration of at least 10 picomolar and no greater than one-tenth of the concentration of the sugar alcohol; or
the sugar alcohol is heptane-1,2,3,4,5,6,7-heptol, and the liquid phase comprises 1,2,3,5,6,7-hexahydroxyheptane-4-oxide, 1,2,4,5,6,7-hexahydroxyheptane-3-oxide, 1,3,4,5,6,7-hexahydroxyheptane-2-oxide, and 2,3,4,5,6,7-hexahydroxyheptane-1-oxide at a combined concentration of at least 10 picomolar and no greater than one-tenth of the concentration of the sugar alcohol.

10. A composition, comprising a liquid phase that comprises propane-1,2-diol at a concentration of at least 1 molar, 1-hydroxypropane-2-oxide and 2-hydroxypropane-1-oxide at a combined concentration of at least 10 nanomolar and no greater than 100 millimolar, a cannabinoid anion at a concentration of at least 1 millimolar and no greater than 500 millimolar, a cannabinoid molecule at a concentration of at least 100 nanomolar and no greater than 50 millimolar, and a cation at a concentration of at least 500 micromolar, wherein:
the liquid phase is miscible with water;
the 1-hydroxypropane-2-oxide, the 2-hydroxypropane-1-oxide, the cannabinoid anion, the cannabinoid molecule, and the cation are solutes that are dissolved in the liquid phase;
the cannabinoid molecule has an acid dissociation constant in water of at least 50 femtomolar and no greater than 50 nanomolar for conversion of the cannabinoid molecule into the cannabinoid anion;
the liquid phase comprises the cannabinoid anion at a greater molar concentration than the cannabinoid molecule;
the cation is a metal cation or an ammonium cation; and
the liquid phase comprises the cation and the cannabinoid anion at a molar ratio of at least 1:3 and no greater than 1000:1.

11. The composition of claim 10, wherein the liquid phase comprises the propane-1,2-diol at a concentration of at least 10 molar; and the liquid phase comprises the 1-hydroxypropane-2-oxide and the 2-hydroxypropane-1-oxide at a combined concentration of at least 100 nanomolar.

12. A composition, comprising a liquid phase that comprises propane-1,2,3-triol at a concentration of at least 1 molar, 1,3-dihydroxypropane-2-oxide and 2,3-dihydroxypropane-1-oxide at a combined concentration of at least 10 nanomolar and no greater than 100 millimolar, a cannabinoid anion at a concentration of at least 1 millimolar and no greater than 500 millimolar, a cannabinoid molecule at a concentration of at least 100 nanomolar and no greater than 50 millimolar, and a cation at a concentration of at least 500 micromolar, wherein:
the liquid phase is miscible with water;
the 1,3-dihydroxypropane-2-oxide, the 2,3-dihydroxypropane-1-oxide, the cannabinoid anion, the cannabinoid molecule, and the cation are solutes that are dissolved in the liquid phase;
the cannabinoid molecule has an acid dissociation constant in water of at least 50 femtomolar and no greater than 50 nanomolar for conversion of the cannabinoid molecule into the cannabinoid anion;
the liquid phase comprises the cannabinoid anion at a greater molar concentration than the cannabinoid molecule;
the cation is a metal cation or an ammonium cation; and
the liquid phase comprises the cation and the cannabinoid anion at a molar ratio of at least 1:3 and no greater than 1000:1.

13. The composition of claim 12, wherein the liquid phase comprises the propane-1,2,3-triol at a concentration of at least 10 molar; and the liquid phase comprises the 1,3-dihydroxypropane-2-oxide and the 2,3-dihydroxypropane-1-oxide at a combined concentration of at least 100 nanomolar.

14. The composition of any one of claims 10-13, wherein the liquid phase comprises ethanol, water, ethoxide, and hydroxide; and the liquid phase comprises the ethanol at a greater concentration by mass than the water.

15. The composition of any one of claims 10-14, wherein the liquid phase comprises ethanol at a concentration of at least 3 molar, and the liquid phase comprises ethoxide at a concentration of at least 30 nanomolar and no greater than one-tenth of the concentration of the ethanol.

16. The composition of any one of claims 10-15, wherein the liquid phase comprises water at a concentration of at least 10 millimolar and no greater than 5 molar.

17. The composition of any one of claims 1-16, wherein the cannabinoid anion has the general structure I, II, or III 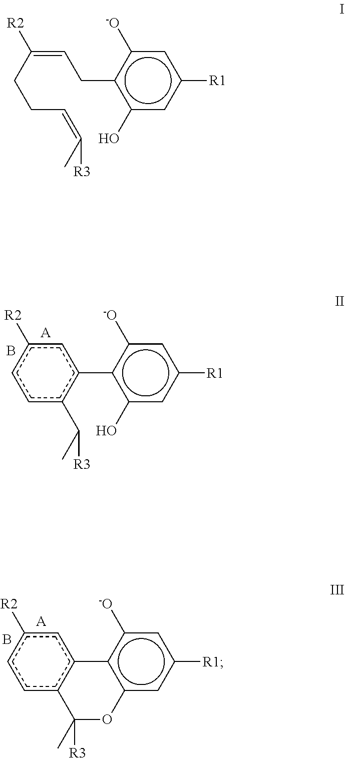
R1 is selected from hydro; a straight or branched C1-C12 alkyl that is optionally substituted with hydroxy, phenyl, a cycloalkyl, or a halogen; a straight or branched C2-C12 alkenyl that is optionally substituted with hydroxy, phenyl, a cycloalkyl, or a halogen; and a straight or branched C2-C12 alkynyl that is optionally substituted with hydroxy, phenyl, a cycloalkyl, or a halogen;
R2 is selected from hydro; methyl; methylidene, hydroxy; hydroxymethyl; fluoromethyl; chloromethyl; bromomethyl; iodomethyl; oxo; formyl; methoxycarbonyl; ethoxycarbonyl; and (2-propoxy)carbonyl;
R3 is selected from a hydro; straight C1-C3 alkyl that is optionally substituted with hydroxy or a halogen; a straight C1-C3 alkenyl that is optionally substituted with hydroxy or a halogen; and a C2-C3 alkynyl that is optionally substituted with hydroxy or a halogen; and
the dotted lines in general structures II and III depict the bonding pattern of either cyclohexane, phenyl, or a cyclohexene that comprises exactly one double bond, which occurs at either A or B.

18. The composition of claim 17, wherein R1 is hydro; methyl; ethyl; propyl; butyl; pentyl; hexyl; heptyl; octyl; nonyl; decyl; prop-2-yl; but-2-yl; pent-2-yl; hex-2-yl; hept-2-yl; octan-2-yl; nonan-2-yl; decan-2-yl; 2-methylpropyl; 2-methylbutyl; 2-methylpentyl; 2-methylhexyl; 2-methylheptyl; 2-methyloctyl; 2-methylnonyl; 2-methyldecyl; 2-methylprop-2-yl; 2-methylbut-2-yl; 2-methylpent yl; 2-methylhex-2-yl; 2-methylhept-2-yl; 2-methyloctan-2-yl; 2-methylnonan-2-yl; 2-methyldecan-2-yl; 3-methylbut-2-yl; 3-methylpent-2-yl; 3-methylhex-2-yl; 3-methylhept-2-yl; 3-methyloctan yl; 3-methylnonan-2-yl; 3-methyldecan-2-yl; 2,3-dimethylbut-2-yl; 2,3-dimethylpent-2-yl; 2,3-dimethylhex-2-yl; 2,3-dimethylhept-2-yl; 2,3-dimethyloctan-2-yl; 2,3-dimethylnonan-2-yl; 2,3-dimethyldecan-2-yl; cyclopropyl; 1-methylcyclopropyl; 1-ethylcyclopropyl; 1-propylcyclopropyl; 1-butylcyclopropyl; 1-pentylcyclopropyl; 1-hexylcyclopropyl; 1-heptylcyclopropyl; 1-octylcyclopropyl; 1-nonylcyclopropyl; cyclobutyl; 1-methylcyclobutyl; 1-ethylcyclobutyl; 1-propylcyclobutyl; 1-butylcyclobutyl; 1-pentylcyclobutyl; 1-hexylcyclobutyl; 1-heptylcyclobutyl; 1-octylcyclobutyl; cyclopentyl; 1-methylcyclopentyl; 1-ethylcyclopentyl; 1-propylcyclopentyl; 1-butylcyclopentyl; 1-pentylcyclopentyl; 1-hexylcyclopentyl; 1-heptylcyclopentyl; cyclohexyl; 1-methylcyclohexyl; 1-ethylcyclohexyl; 1-propylcyclohexyl; 1-butylcyclohexyl; 1-pentylcyclohexyl; 1-hexylcyclohexyl; ethenyl; prop-1-enyl; but-1-enyl; pent-1-enyl; hex-1-enyl; hept-1-enyl; octan-1-enyl; nonan-1-enyl; decan-1-enyl; ethynyl; prop-1-ynyl; but-1-ynyl; pent-1-ynyl; hex-1-ynyl; hept-1-ynyl; octan-1-ynyl; nonan-1-ynyl; decan-1-ynyl; 2-phenylethyl; 2-phenylprop-2-yl; adamant-1-yl; adamant-2-yl; 6-halohex-2-enyl; 6-halohex-2-ynyl; or 2-methyl-6-halohex-2-yl.

19. The composition of claim 17 or 18, wherein R1 is propyl or pentyl.

20. The composition of any one of claims 17-19, wherein either:
R2 is hydro, methyl, hydroxy, or hydroxymethyl; or
the cannabinoid anion has the general structure II or III; R2 is oxo; and the dotted lines in general structures II and III depict the bonding pattern of cyclohexane.

21. The composition of any one of claims 17-20, wherein R2 is methyl.

22. The composition of any one of claims 17-21, wherein either:
the cannabinoid anion has the general structure I or III, and R3 is methyl; or
the cannabinoid anion has the general structure II, and R3 is methylidene.

23. The composition of any one of claims 17-21, wherein the cannabinoid anion has the general structure II or III; and R3 is methyl, 3-hydroxypropyl, 3-hydroxyprop-1-enyl, or 3-hydroxyprop-1-ynyl.

24. The composition of any one of claims 1-23, wherein the cannabinoid anion is 2-geranyl-3-hydroxy-5-pentylphenolate.

25. The composition of any one of claims 1-23, wherein the cannabinoid anion is 2-geranyl hydroxy-5-propylphenolate.

26. The composition of any one of claims 1-23, wherein the cannabinoid anion is 3-hydroxy-2-(6-isopropenyl-3-methylcyclohex-2-enyl)-5-pentylphenolate.

27. The composition of any one of claims 1-23, wherein the cannabinoid anion is 3-hydroxy-2-(6-isopropenyl-3-methylcyclohex-2-enyl)-5-propylphenolate.

28. The composition of any one of claims 1-23, wherein the cannabinoid anion is 6,6,9-trimethyl-3-pentyl-6a,7,8,10a-tetrahydro-6H-benzo[c]chromene-1-oxide.

29. The composition of any one of claims 1-23, wherein the cannabinoid anion is 6,6,9-trimethyl-3-propyl-6a,7,8,10a-tetrahydro-6H-benzo[c]chromene-1-oxide.

30. The composition of any one of claims 1-23, wherein the cannabinoid anion is 6,6,9-trimethyl-3-pentyl-6H-benzo[c]chromene-1-oxide.

31. The composition of any one of claims 1-23, wherein the cannabinoid anion is 6,6,9-trimethyl-3-propyl-6H-benzo[c]chromene-1-oxide.

32. The composition of any one of claims 1-31, wherein the cation is lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); copper (II) cation (“Cu++”); ammonium (“NH₄+”); protonated ethanolamine; choline; protonated lysine; protonated arginine; or protonated sphingosine.

33. The composition of any one of claims 1-32, wherein the cation is sodium cation.

34. The composition of any one of claims 1-32, wherein the cation is potassium cation.

35. The composition of any one of claims 1-34, wherein the cannabinoid molecule has a solubility in the liquid phase that is less than the concentration of the cannabinoid anion in the liquid phase.

36. A composition prepared from a starting composition according to any one of claims 1-35, comprising a liquid phase that comprises a cannabinoid molecule and a cation, wherein:
the cannabinoid molecule and the cation are solutes that are dissolved in the liquid phase;
the composition is prepared by contacting the starting composition with a Brønsted acid to convert the cannabinoid anion of the starting composition into the cannabinoid molecule;
the liquid phase comprises the cation and the cannabinoid molecule at a molar ratio of at least 1:3; and
the cannabinoid molecule has a solubility in the liquid phase that is less than the concentration of the cannabinoid molecule that is dissolved in the liquid phase such that the liquid phase is supersaturated with the cannabinoid molecule.

37. The composition of any one of claims 1-36 for use as a medicament.

38. The composition of any one of claims 1-37, wherein the composition is formulated to convert the cannabinoid anion into the cannabinoid molecule ex vivo prior to administering the composition to a subject.

39. The composition of any one of claims 1-37, wherein the composition is formulated to convert the cannabinoid anion into the cannabinoid molecule in situ subsequent to administering the composition to a subject.

40. The composition of any one of claims 1-39, wherein the composition is formulated for oral administration to a subject; the composition is formulated to allow the conversion of the cannabinoid anion into the cannabinoid molecule before the cannabinoid anion reaches the stomach of the subject; and the composition is formulated to allow absorption of the cannabinoid molecule by the epithelial lining of the gastrointestinal tract between the lips and the stomach, excluding the stomach and the outer surfaces of the lips, and including the esophagus and the inner surfaces of the lips.

41. The composition of any one of claims 1-39, wherein the composition is formulated for topical administration to a subject.

42. A method to administer a cannabinoid, comprising providing a composition according to any one of claims 1-41, and administering the composition to a subject.

43. The method of claim 42, comprising converting the cannabinoid anion into the cannabinoid molecule ex vivo prior to the administering.

44. The method of claim 42, comprising converting the cannabinoid anion into the cannabinoid molecule in situ subsequent to the administering.

45. The method of any one of claims 42-44, wherein the administering is oral administering; the composition is formulated to allow the conversion of the cannabinoid anion into the cannabinoid molecule before the cannabinoid anion reaches the stomach of the subject; and the composition is formulated to allow absorption of the cannabinoid molecule by the epithelial lining of the gastrointestinal tract between the lips and the stomach, excluding the stomach and the outer surfaces of the lips, and including the esophagus and the inner surfaces of the lips.

46. The method of any one of claims 42-44, wherein the administering is topical administering.

47. The composition or method of any one of claims 38-46, wherein the subject is a rodent, lagomorph, feline, canine, porcine, ovine, caprine, lama, vicugna, bovine, equine, or primate.

48. The composition or method of any one of claims 38-47, wherein the subject is human.

49. The composition or method of any one of claims 1-48, wherein the composition is effective to prophylactically prevent or treat muscle cramping, muscle spasms, restless-legs syndrome, nystagmus, a dyskinetic movement disorder, tremor, seizures, epilepsy, muscular dystrophy, or inclusion body myositis.

50. The composition or method of any one of claims 1-48, wherein the composition is effective to either arrest or reduce the severity of an active seizure.

51. The composition or method of any one of claims 1-48, wherein the composition is effective to reduce blood pressure.

52. The composition or method of any one of claims 1-48, wherein the composition is effective to prophylactically prevent or treat prehypertension or hypertension.

53. The composition or method of any one of claims 1-48, wherein the composition is effective to treat attention deficit hyperactivity disorder (“ADHD”), autism or an autism spectrum disorder, Asperger syndrome, fragile X syndrome, Down syndrome, a pervasive developmental disorder not otherwise specified (“PDD-NOS”), a childhood disintegrative disorder, or Tourette's syndrome.

54. The composition or method of any one of claims 1-48, wherein the composition is effective to treat anxiety, post-traumatic stress disorder (“PTSD”), depression, bipolar disorder, obsessive-compulsive disorder, schizophreniform disorder, schizophrenia, or psychosis.

55. The composition or method of any one of claims 1-48, wherein the composition is effective to treat pain or inflammation.

56. The composition or method of any one of claims 1-48, wherein the composition is effective to treat asthma.

57. The composition or method of any one of claims 1-48, wherein the composition is effective to treat an autoimmune disorder.

58. The composition or method of any one of claims 1-48, wherein the composition is effective to treat arthritis, ankylosing spondylitis, an inflammatory autoimmune-mediated arthritis, rheumatoid arthritis, psoriatic arthritis, psoriasis, plaque psoriasis, lupus, Sjogren's syndrome, inflammatory bowel disease, Crohn's disease, or ulcerative colitis.

59. The composition or method of any one of claims 1-48, wherein the composition is effective to treat a neurodegenerative disease or neuropathy.

60. The composition or method of any one of claims 1-48, wherein the composition is effective to treat Parkinson's Disease, and treating the Parkinson's Disease comprises treating Parkinsonian tremor.

61. The composition or method of any one of claims 1-48, wherein the composition is effective to treat multiple sclerosis, mild cognitive impairment, Alzheimer's Disease, amyotrophic lateral sclerosis (“ALS”), or Huntington's disease.

62. The composition or method of any one of claims 1-48, wherein the composition is effective to treat obesity, metabolic syndrome, or diabetes mellitus.

63. The composition or method of any one of claims 1-48, wherein the composition is effective to treat a viral infection or a bacterial infection.

64. The composition or method of any one of claims 1-48, wherein the composition is effective to treat an infection caused by Escherichia coli, Klebsiella pneumoniae, Pseudomonas aeruginosa, Acinetobacter baumannii, Moraxella catarrhalis, Legionella pneumophila, Staphylococcus aureus, Streptococcus pneumoniae, Enterococcus faecium, Clostridioides difficile, Mycobacterium tuberculosis, Neisseria gonorrhoeae, Cutibacterium acnes, or COVID-19.
